(19) United States
(12) Patent Application Publication
(10) Pub. No.: US 2011/0159582 A1
(43) Pub. Date: Jun. 30, 2011

(54) Apparatus for the growth of artificial organic items, especially human or animal skin

(75) Inventors: Meir Israelowitz, Toronto (CA); Herbert Peter von Schroeder, Toronto (CA); Syed Rizvi, Lake Mary, FL (US); Christoph Gille, Hohen Nenedorf (DE); Chris Scott Holm, Wuppertal (DE)

(73) Assignee: BIOMIMETICS TECHNOLOGIES INC, Toronto (CA)

(21) Appl. No.: 12/655,238

(22) Filed: Dec. 28, 2009

(51) Int. Cl. C12M 3/00

(52) U.S. Cl. 435/293.1; 435/303.1

(57) ABSTRACT

The invention relates to an Apparatus for the growth of artificial organic items, particularly human or animal skin. The growth of skin is possible if a slowly stretched piece of skin or skin substitute is in contact with the nutrient fluid only on its inner side, and in contact with a gas, especially with air, on its outer side.

### BACKGROUND OF THE INVENTION

### Modify Dec. 26, 2010

The WO 2006/088029 describes and shows a bioreactor in which cells grow in a laminar flow of a nutrient fluid (Weyand B, Israelowitz M, von Schroeder H, Vogt P M. 2009. Fluid dynamics in bioreactor design: considerations for the theoretical and practical approach. In: Kasper C, van Griensven M and Portner R., eds. Bioreactor Systems for Tissue Engineering. Advances in Biochemical Engineering/Biotechnology 112. Heidleberg: Springer Berlin; p 251-268; Israelowitz M, Rizvi S, Weyand B, von Schroeder H P. 2010. Development of a laminar flow bioreactor using computational fluid dynamics. J. Healthcare and Engineering [In press]). This growth is insufficient to create human or animal skin. Skin is a flat item/organ with profound differences between its inner and outer sides and organized in layers. In order to produce a larger piece of skin, it is necessary to stretch a piece of lesser dimension, and allow for the accumulation and growth of cells during the stretching process of the original skin. This is not possible in the WO 2006/088029 apparatus.

### OBJECT OF THE INVENTION

The inventors have found that the growth of skin (Weyand B, Reimers K, Vogt P M. 2010. Influences of extracellular matrix properties and flow shear stresses on stem cell shape in a three-dimensional dynamic environment, IFMBE Proceedings, 2010) is possible if a slowly stretched piece of skin is in contact with the nutrient fluid only on its inner side, and in contact with a gas, especially with air, on the outer side of the skin.

### DESCRIPTION OF THE INVENTION

Therefore, the apparatus of the invention consists of a chamber that is only partly filled with a flowing nutrient fluid. This chamber contains a holder for keeping the organic item such as skin on the surface of the nutrient fluid and includes items to stretch the skin. The chamber has an inlet and an outlet for the nutrient fluid under the surface of the skin; and, it has another inlet and an outlet for a gas, such as air, above the surface of the skin. With this apparatus, it is possible to enlarge a piece of skin by stretching and allowing for the growth of the tissue with the supply of cells and nutrient fluid such that there is no essential enlargement or reduction of the thickness of the skin.

To ensure that the inner side of the skin or another organic item is always in contact with the nutrient fluid, the organic item, in this device, is fastened on the under side of the holder in such a way as to have contact with the surface of the nutrient fluid (Kuhbier J W, Weyand B, Radtke C, Vogt P M, Kasper C, and Reimers K, 2010. Isolation, Characterization, Differentiation and Application of Adipose-Derived Stem Cells, Adv Biochem Eng Biotechnol, (PMID: 20091288); Kuhbier J W, Weyand B, Sorg H, Radtke C, Vogt P M, and Reimers K, 2010. [Stem cells from fatty tissue: A new resource for regenerative medicine?], Chirurg, 81: 826-832). In order to stretch the skin, this device has moveable clamps that hold the organic item.

The holder is made from sheet metal, with one or more openings covered with the organic item. It is maintained in a completely horizontal position and parallel to the surface of the fluid over its dimensions and in contact with the nutrient fluid.

The apparatus has a holder with more than one opening. Each opening can be covered with one organic item.

It is beneficial if the apparatus has a valve in the fluid inlet, that consists of two displaceable sheets with openings one upon another to create a laminar flow of the nutrient fluid.

This apparatus is advantageously part of a circuit consisting of pipes for the transport of the nutrient fluid, a pump, and a source of nutrients mixed in the flow.

To control the function of this apparatus, it is provided with glass portals to observe the flow, and growth of the organic item.

This apparatus is supplied with adjusters for the exact horizontal position of the holder.

### BACKGROUND ART

1. US Patent Application 20090061501 Israelowitz, et al.
2. EU Patent 08018427.8 Israelowitz, et al.

### REFERENCES

1. Weyand B, Israelowitz M, von Schroeder H, Vogt P M. 2009. Fluid dynamics in bioreactor design: considerations for the theoretical and practical approach. In: Kasper C, van Griensven M and Portner R., eds. Bioreactor Systems for Tissue Engineering. Advances in Biochemical Engineering/Biotechnology 112. Heidleberg: Springer Berlin; p 251-268.
2. Israelowitz M, Rizvi S, Weyand B, Vogt P, von Schroeder H P. 2011. Development of a laminar flow bioreactor using computational fluid dynamics, J Eng and Health Care [In press].
3. Weyand B, Reimers K, Vogt P M. 2010. Influences of extracellular matrix properties and flow shear stresses on stem cell shape in a three-dimensional dynamic environment, IFMBE Proceedings.
4. Kuhbier J W, Weyand B, Radtke C, Vogt P M, Kasper C, and Reimers K, 2010. Isolation, Characterization, Differentiation and Application of Adipose-Derived Stem Cells, Adv Biochem Eng Biotechnol, (PMID: 20091288)
5. Kuhbier J W, Weyand B, Sorg H, Radtke C, Vogt P M, and Reimers K, 2010. [Stem cells from fatty tissue: A new resource for regenerative medicine?], Chirurg, 81: 826-832.

### DESCRIPTION OF THE DRAWINGS

### Modify Dec. 26, 2010

FIG. 1 shows a block diagram of the installation for the culture and growth of cells to a three dimensional tissue.

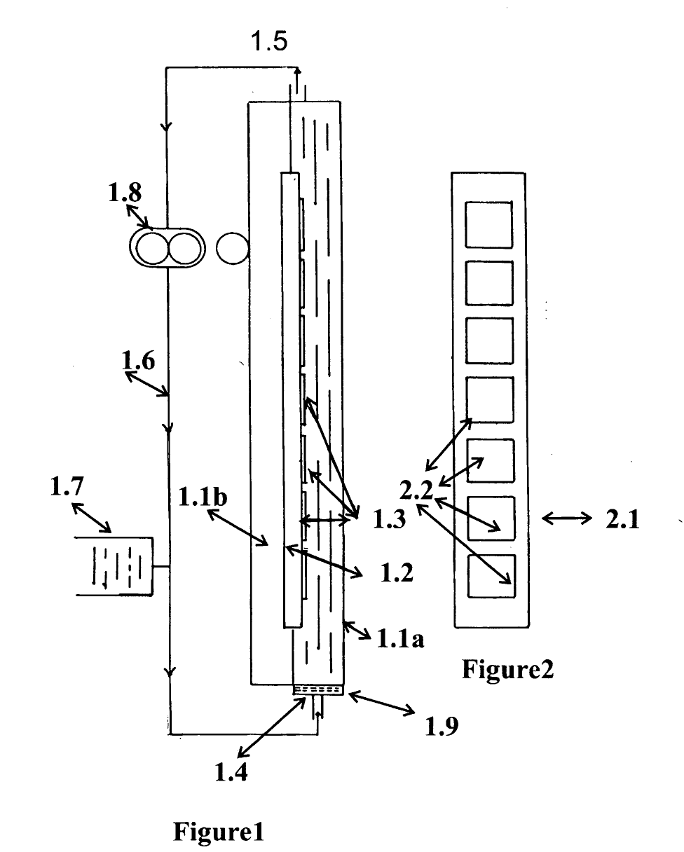

FIG. 2 shows a holder for several parts of growing cell masses.

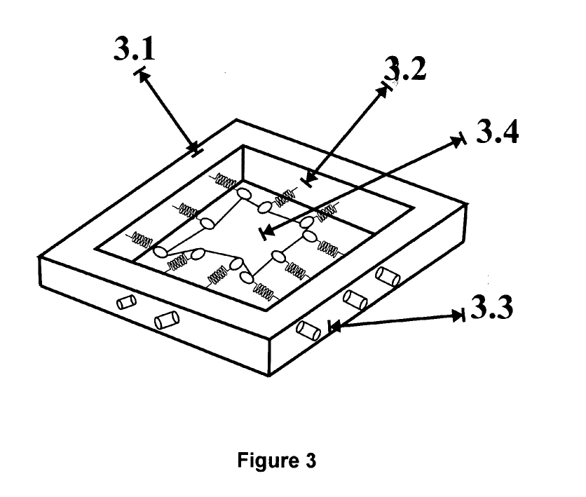

FIG. 3 shows an individual cell with holders, spring to stretch and access to detector to produce the stretch

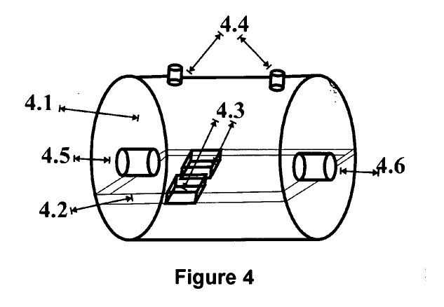

FIG. 4 shows a perspective of the device

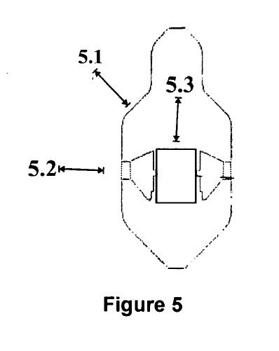

FIG. 5 shows cell integrated to the laminar flow system

### DETAILED DESCRIPTION OF THE DRAWINGS

### Modified Dec. 26, 2010

The block diagram of FIG. 1.0 shows in a cross-section the inside of the bio-reactor (1.1a) with a holder (1.2) for tissue samples, and cell masses (1.3) which are situated in a horizontal position. The part of the bio-reactor (1.1a) under the holder (1.2) is filled with the nutrient fluid (lined section) (2.1) contacting the under site of the samples (1.3) and cell masses. The upper part of the bio-reactor (1.1b) is filled with gas, such as air.

The holder FIG. 2.1 is provided with rectangular holes (2.2) on the underside covered with the tissue samples (1.3) and cell masses. In this way the nutrient fluid contacts only the under-side of the samples but not the upper side which is in contact with the gas.

Each of the rectangular holes (1.4) are surrounded with clamps for holding the samples of cell masses and tools for moving the clamps in a direction to stretch and enlarge the samples (1.3) of cell masses. The clamps and tools are not shown.

The nutrient fluid is circulated by a pump (1.5) through pipes (1.6).

A supply tank (1.7) with nutrient fluid is connected to the system of pipes (1.6) to refill the stock of the fluid.

Because the flow of fluid has to be a laminar flow at the entrance to the under part of the bio-reactor (1.1a), a valve (1.8) is installed at this entrance. This valve consists of two disks (1.9), perforated in the same way and at the same locations. These disks (1.9) can be rotated one against the other in small degrees to change the rate of flow and circulation. The result of this small rotation of the two perforated disks (1.9), one against the other is the control of the upper level of the fluid and the kind of flow that has to be a laminar flow. The thickness of the holder (2.1) gives the range of the surface of fluid.

FIG. 3 shows a single cell holder (3.1), with holders attached to the springs for stretching (3.2). Each individual holder and the spring is controlled by individual sensors for the stretching (3.3) and the organic material is gripped by each individual holder with the stretcher.

FIG. 4 shows the device divided in a section with gas (i.e. air) (4.1) and a section with medium (4.2). The single cells are designed for holding and stimulating of the organic material (4.3). The gas inlet and outlet is shown in (4.4). The medium inlet is shown in (4.5) and the medium outlet shown in (4.6).

FIG. 5 shows the cell integrated to a single cell holder (5.1). The dot line show the bypass, which is opened or closed to release the pressures and control shear stresses (5.2) and the single holder is shown in (5.3).

1. Apparatus for the cell differentiation specifically for human or animal skin the device which is divided in two sections one section filled with a flowing nutrient fluid and one section which is filled with gas in this chamber there is a holder to keep the organic item on the surface of the nutrient fluid where the chamber has an inlet and an outlet for the nutrient fluid and under the surface of the nutrient fluid it has between this inlet and outlet another entrance for the gas section above the surface of the fluid in the same device.

2. Apparatus of claim 1 the organic item is fastened on the underside of the holder in a way to have contact with the surface of the nutrient fluid.

3. Apparatus of claim 1 the holder in the chamber carries moveable clamps holding the organic item to created mechanical stresses to support cell differentiation, cell migration and cell differentiation and extracellular matrix deposition for example collagen synthesis.

4. Apparatus of claim 1 the holder support the mobile clamps with one or more openings covered with the organic item.

5. Apparatus of claim 1 with two openings for a holder covered with cell where one part of the holder is exposed to gas phase and the other half is exposed to a medium.

6. Apparatus of claim 1 in the inlet outlet with a differential pressure sensor for flow control.

7. Apparatus of claim 1 being part of a circuit consisting of pipes for the transport of the nutrient fluid, a pump and a source of nutrients mixed in the flow for one section of the devices and for the other part of the devices transport is by external inlet with outlet for gas entering this section of the chamber.

8. Apparatus of claim 1 provided with glass portals to see and observe the kind of flow and the growth of the organic item.

9. Apparatus according claim 1 with adjusters for the exact horizontal position of the holder.
